(19) United States
(12) Patent Application Publication
(10) Pub. No.: US 2020/0367533 A1
(43) Pub. Date: Nov. 26, 2020

(54) Method of fermenting Rosa sterilis var leioclada

(71) Applicants: Chang-Lu Hsu, Kaohsiung (TW); Yi-Jinn Chen, Kaohsiung (TW)

(72) Inventors: Chang-Lu Hsu, Kaohsiung (TW); Yi-Jinn Chen, Kaohsiung (TW)

(21) Appl. No.: 16/991,051

(22) Filed: Aug. 12, 2020

(51) Int. Cl. A23L 2/38

(52) U.S. Cl. CPC A23L 2/382; C12N 1/16; A23Y 2220/17; A23Y 2300/55

(57) ABSTRACT

A method of fermenting Rosa sterilis var. leioclada includes mixing a Lactobacillus casei strain, a Bifidobacterium longum strain, and a Saccharomyces cerevisiae strain to form a microbial mixture wherein a cell number ratio of the Lactobacillus casei strain, the Bifidobacterium longum strain and the Saccharomyces cerevisiae strain is 0.3-1:0.2-0.9:1-1.8; preparing a fruit sample of Rosa sterilis var. leioclada; and fermenting the fruit sample of Rosa sterilis var. leioclada with the microbial mixture at 22-33° C. for 6-15 days to obtain a fermented juice wherein a total concentration of the Lactobacillus casei strain, the Bifidobacterium longum strain, and the Saccharomyces cerevisiae strain in a mixture solution containing the microbial mixture and the fruit sample of Rosa sterilis var. leioclada is between 1.5×10⁷ and 3.5×10⁷ CFU/mL; and a volumetric percentage of the microbial mixture is 10-25% of a volume of the mixture solution.

### CROSS-REFERENCE TO RELATED APPLICATION

This application is a continuation-in-part (CIP) of U.S. patent application Ser. No. 15/843,779, filed Dec. 15, 2017, now abandoned, which is incorporated herein by reference in its entirety.

### BACKGROUND OF THE INVENTION

### 1. Field of the Invention

The invention relates to fermentation and more particularly to a method of fermenting Rosa sterilis var. leioclada to obtain a fermented juice with improved anti-oxidation characteristics.

### 2. Description of Related Art

As the indusexperimentization progresses, more and more pollutants are found in the environment. People come into intact with the pollutants every day, resulting in free radicals found in the human body, which are the main causes of many civilized diseases in modern times. Therefore, people nowadays pay more and more attention to anti-oxidation to remove the free radicals found in the human bodies.

Taking fruits of Rosa sterilis var. leioclada (Rosaceae) is a way to absorb antioxidants. However, in order to obtain enough antioxidants, a lot of fruits should be taken. In light of this, it is necessary to develop a method of fermenting Rosa sterilis var. leioclada to obtain a fermented juice with improved anti-oxidation ability.

### SUMMARY OF THE INVENTION

It is therefore one object of the invention to provide a method of fermenting Rosa sterilis var. leioclada, comprising the steps of mixing a Lactobacillus casei strain, a Bifidobacterium longum strain, and a Saccharomyces cerevisiae strain to form a microbial mixture wherein a cell number ratio of the Lactobacillus casei strain, the Bifidobacterium longum strain and the Saccharomyces cerevisiae strain is 0.3-1:0.2-0.9:1-1.8; preparing a fruit sample of Rosa sterilis var. leioclada; and fermenting the fruit sample of Rosa sterilis var. leioclada with the microbial mixture at 22-33° C. for 6-15 days to obtain a fermented juice wherein a total concentration of the Lactobacillus casei strain, the Bifidobacterium longum strain, and the Saccharomyces cerevisiae strain in a mixture solution containing the microbial mixture and the fruit sample of Rosa sterilis var. leioclada is between 1.5×10⁷ and 3.5×10⁷ CFU/mL; and a volumetric percentage of the microbial mixture is 10-25% of a volume of the mixture solution.

The above and other objects, features and advantages of the invention will become apparent from the following detailed description taken with the accompanying drawings.

### BRIEF DESCRIPTION OF THE DRAWINGS

FIG. 1 is a flow chart illustrating a method of fermenting Rosa sterilis var. leioclada according to the invention;

FIG. 1a depicts a bar chart illustrating ABTS+ clearance rate of the fermented juices of groups A1 to A4 as well as the unfermented juice of group A5 in experiment A;

FIG. 1b depicts a bar chart illustrating carotenoid level of the fermented juices of groups A1 to A4 as well as the unfermented juice of group A5 in experiment A;

FIG. 1c depicts a bar chart illustrating vitamin C level of the fermented juices of groups A1 to A4 as well as the unfermented juice of group A5 in experiment A;

FIG. 1d depicts a bar chart illustrating SOD activity of the fermented juices of groups A1 to A4 as well as the unfermented juice of group A5 in experiment A;

FIG. 2a depicts a bar chart illustrating ABTS+ clearance rate of the fermented juices of groups B1 to B2 as well as the unfermented juice of group B3 in experiment B;

FIG. 2b depicts a bar chart illustrating vitamin C level of the fermented juices of groups B1 to B2 as well as the unfermented juice of group B3 in experiment B;

FIG. 2c depicts a bar chart illustrating carotenoid level of the fermented juices of groups B1 to B2 as well as the unfermented juice of group B3 in experiment B;

FIG. 2d depicts a bar chart illustrating SOD activity of the fermented juices of groups B1 to B2 as well as the unfermented juice of group B3 in experiment B;

FIG. 3a depicts a bar chart illustrating ABTS+ clearance rate of the fermented juices of groups C1 to C2 as well as the unfermented juice of group C3 in experiment C;

FIG. 3b depicts a bar chart illustrating vitamin C level of the fermented juices of groups C1 to C2 as well as the unfermented juice of group C3 in experiment C;

FIG. 3c depicts a bar chart illustrating carotenoid level of the fermented juices of groups C1 to C2 as well as the unfermented juice of group C3 in experiment C;

FIG. 3d depicts a bar chart illustrating SOD activity of the fermented juices of groups C1 to C2 as well as the unfermented juice of group C3 in experiment C;

FIG. 4a depicts a bar chart illustrating ABTS+ clearance rate of the fermented juices of groups D1 to D2 as well as the unfermented juice of group D3 in experiment D;

FIG. 4b depicts a bar chart illustrating vitamin C level of the fermented juices of groups D1 to D2 as well as the unfermented juice of group D3 in experiment D;

FIG. 4c depicts a bar chart illustrating carotenoid level of the fermented juices of groups D1 to D2 as well as the unfermented juice of group D3 in experiment D; and

FIG. 4d depicts a bar chart illustrating SOD activity of the fermented juices of groups D1 to D2 as well as the unfermented juice of group D3 in experiment D.

### DETAILED DESCRIPTION OF THE INVENTION

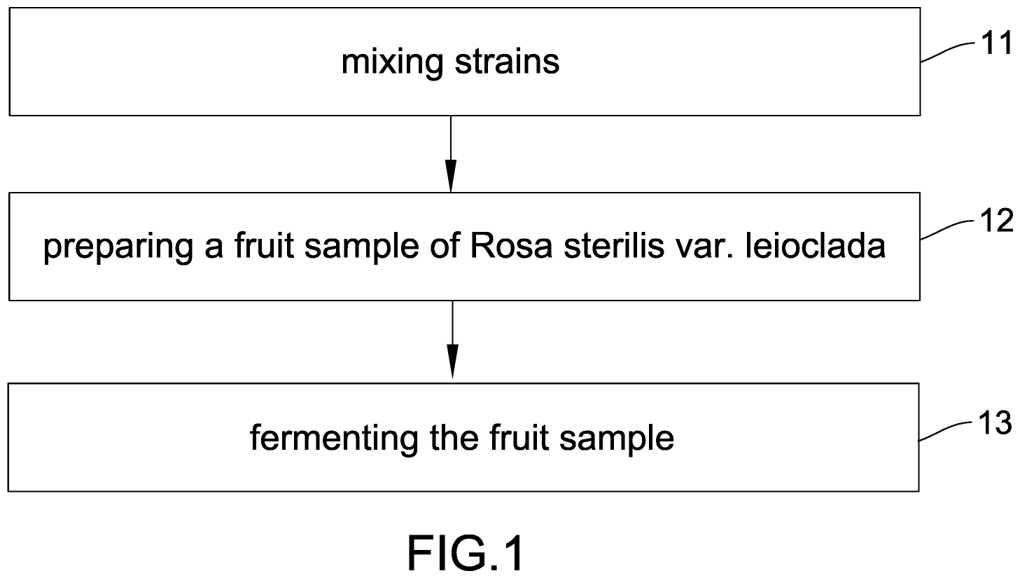

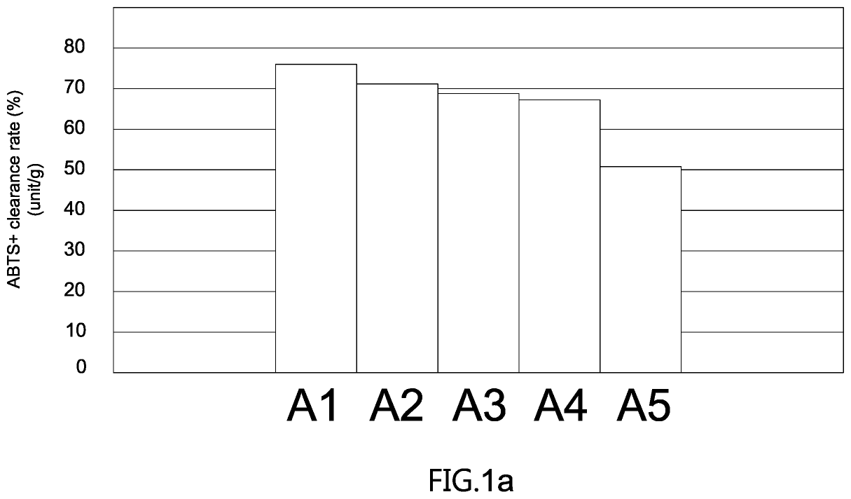

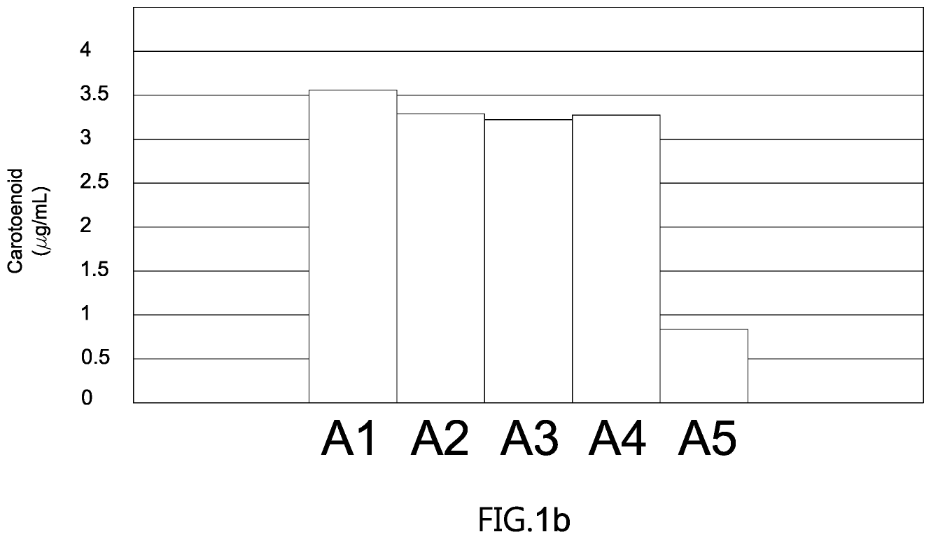

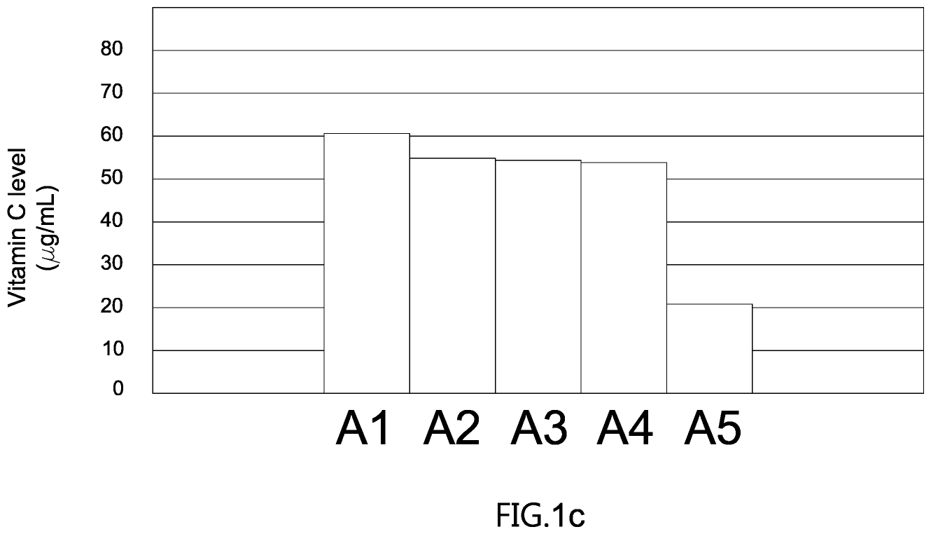

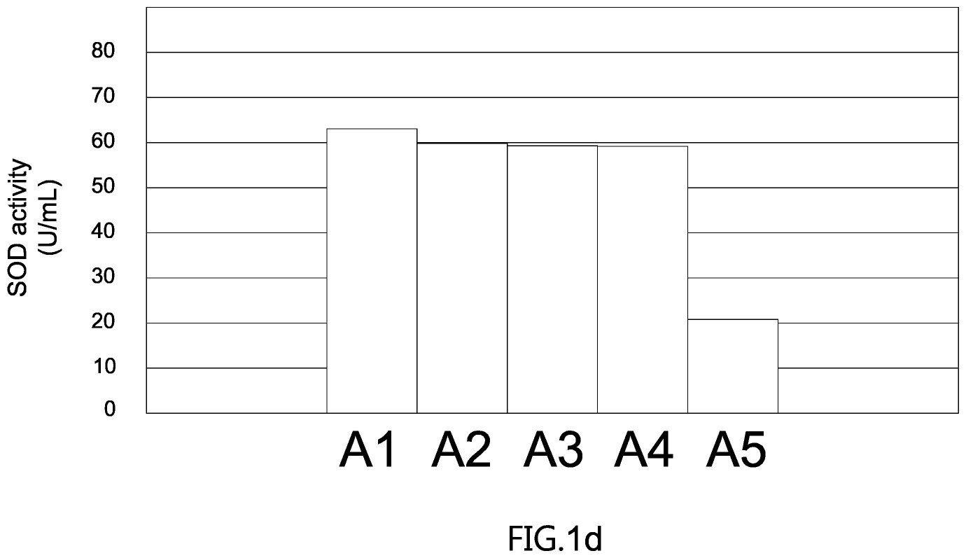

Referring to FIG. 1, a flow chart of a method of fermenting Rosa sterilis var. leioclada in accordance with the invention is illustrated. The method comprises step 11 of mixing a Lactobacillus casei strain, a Bifidobacterium longum strain, and a Saccharomyces cerevisiae strain to form a microbial mixture wherein a cell number ratio of the Lactobacillus casei strain, the Bifidobacterium longum strain and the Saccharomyces cerevisiae strain is 0.3-1:0.2-0.9:1-1.8; step 12 of preparing a fruit sample of Rosa sterilis var. leioclada; and step 13 of fermenting the fruit sample of Rosa sterilis var. leioclada with the microbial mixture at 22-33° C. for 6-15 days to obtain a fermented juice wherein a total concentration of the Lactobacillus casei strain, the Bifidobacterium longum strain, and the Saccharomyces cerevisiae strain in a mixture solution containing the microbial mixture and the fruit sample of Rosa sterilis var. leioclada is between 1.5×10⁷ and 3.5×10⁷ CFU/mL; and a volumetric percentage of the microbial mixture is 10-25% of a volume of the mixture solution.

Preferably, a L. casei strain BCRC 10697, a B. longum strain BCRC 14604 and a S. cerevisiae strain BCRC 20579 can be purchased from the Bioresource Collection and Research Center (BCRC) of the Food Industry Research and Development Institute (FIRDI) of Taiwan for forming the microbial mixture.

The fruit sample can be a whole fruit with pulp, peel and seeds. As an example, the whole fruit can be steamed and softened, and the microbial mixture can infiltrate into the whole fruit and fermentation efficiency can be effectively increased. Alternatively, the fruit sample can be the whole fruit which is incised, such as the whole fruit with an incision formed on the peel of the whole fruit or the whole fruit which is cut in half or sliced into several pieces. Therefore, pulp of Rosa sterilis var. leioclada is exposed to the microbial mixture and fermentation efficiency can be effectively increased. In this embodiment, the fruit sample is selected to be a juice of Rosa sterilis var. leioclada (hereinafter “the juice”) which is obtained by squeezing the whole fruit (with pulp, peel and seeds). Accordingly, compared to using the whole fruit (with pulp, peel and seeds) and the whole fruit which is incised as the fruit sample, using the juice as the fruit sample increases mixing efficiency of the fruit sample and the microbial mixture as well as the fermentation efficiency.

To activate the L. casei strain, the B. longum strain and the S. cerevisiae strain, the L. casei strain, the B. longum strain and the S. cerevisiae strain can preferably be cultured before the fruit sample is fermented by the microbial mixture. With such performance, the L. casei strain, the B. longum strain and the S. cerevisiae strain can become healthier and the fermentation efficiency can thus be improved. In this embodiment, the L. casei strain, the B. longum strain and the S. cerevisiae strain are cultured in a liquid medium separately to prevent from inhibition of growth of one strain due to growth of another strain. The L. casei strain, the B. longum strain and the S. cerevisiae strain are cultured to log phase (logarithmic phase) and a solution of L. casei, a solution of B. longum and a solution of S. cerevisiae are formed respectively. Moreover, the L. casei strain, the B. longum strain and the S. cerevisiae strain can be separately cultured at 37° C. which is the optimal culturing temperature for all of the L. casei strain, the B. longum strain and the S. cerevisiae strain, and thus quality of the L. casei strain, the B. longum strain and the S. cerevisiae strain can be assured. Besides, cell number of the L. casei strain, the B. longum strain and the S. cerevisiae strain can be counted using a hemocytometer, and thus concentrations of the solution of L. casei, the solution of B. longum and the solution of S. cerevisiae can be adjusted to 1×10⁷ CFU/mL. It is worthy to noted that in the microbial mixture, the cell number ratio of the L. casei strain, the B. longum strain and the S. cerevisiae strain is 0.3-1:0.2-0.9:1-1.8, preferably the cell number percentages of the L. casei strain, the B. longum strain and the S. cerevisiae strain is 20-25%, 13-25% and 50-64%, respectively. Thus, the L. casei strain, the B. longum strain and the S. cerevisiae strain can have great interaction efficiency. Preferably, the cell number ratio of the L. casei strain, the B. longum strain and the S. cerevisiae strain is 0.8:0.8:1.6 to form the microbial mixture. That is, the cell number percentages of the L. casei strain, the B. longum strain and the S. cerevisiae strain are 25%, 25% and 50% by cell number of the microbial mixture, respectively. In such performance, the best fermentation efficiency can be achieved.

Moreover, the microbial mixture and the fruit sample can be mixed to obtain a mixture solution. A total concentration of the L. casei strain, the B. longum strain and the S. cerevisiae strain in the mixture solution is between 1.5×10⁷ and 3.5×10⁷ CFU/mL. In addition, a volumetric percentage of the microbial mixture can be 10-25% by volume of the mixture solution. Accordingly, the fermentation process can be processed with enough microbial cells and the fermentation efficiency can be assured.

The fruit sample is then fermented by the microbial mixture at the optimal condition to obtain the fermented juice. In detail, the fruit sample is fermented by the microbial mixture at 22-33° C. for 6-15 days. Moreover, the fruit sample can be fermented by the microbial mixture with or without agitation. As an example, the fruit sample can be fermented by the microbial mixture with agitation at a rotation speed of 80-110 rpm to prevent from the occurrence of precipitation. In this embodiment, the fruit sample is fermented by the microbial mixture at a temperature no greater than 28° C. for 14 days with agitation at the rotation speed of 100 rpm.

To evaluate the fermented juice with improved anti-oxidation ability can be obtained by using the method of fermenting Rosa sterilis var. leioclada according to the invention, the following experiments are carried out by referring to FIGS. 1a to 1d in which FIG. 1a depicts a bar chart illustrating ABTS+ clearance rate of the fermented juices of groups A1 to A4 as well as the unfermented juice of group A5 in experiment A, FIG. 1b depicts a bar chart illustrating carotenoid level of the fermented juices of groups A1 to A4 as well as the unfermented juice of group A5 in experiment A, FIG. 1c depicts a bar chart illustrating vitamin C level of the fermented juices of groups A1 to A4 as well as the unfermented juice of group A5 in experiment A, and FIG. 1d depicts a bar chart illustrating SOD activity of the fermented juices of groups A1 to A4 as well as the unfermented juice of group A5 in experiment A.

In the experiment A, a L. casei strain, a B. longum strain and a S. cerevisiae strain are mixed in the cell number ratio of 0.3-1:0.2-0.9:1-1.8 to obtain the microbial mixture. The juice is then fermented by the microbial mixture at a temperature of 22-33° C. for 6-15 days to form the fermented juice of group (i.e., experimental group) A1.

Moreover, a Lactobacillus acidophilus strain, a B. longum strain and a S. cerevisiae strain are mixed in a cell number ratio of 0.8:0.8:1.6 to obtain another microbial mixture. The juice is then fermented by the microbial mixture at a temperature of 22-33° C. for 6-15 days to form the fermented juice of group (i.e., a first control group) A2. In short, the difference between the groups is that L. casei strain is used in the group A1 and Lactobacillus acidophilus strain is used in the group A2. Thus, it is possible of observing whether there is different effect between the group A1 and the group A2.

Further, a Bifidobacterium bifidum strain, a Saccharomyces boulardii strain and a Lactobacillus acidophilus strain are mixed in a cell number ratio of 0.8:0.8:1.6 to obtain another microbial mixture. The juice is then fermented by the microbial mixture at a temperature of 22-33° C. for 6-15 days to form the fermented juice of group (i.e., a second control group) A3. Thus, it is possible of observing whether there is different effect between the group A1 and the group A3 using three different strains.

Furthermore, a Lactobacillus casei strain, a Bifidobacterium longum strain, a Saccharomyces cerevisiae strain and a Lactobacillus bulgaricus strain are mixed in a cell number ratio of 1-2:1-2:1-1.8:0.4-0.8 to obtain another microbial mixture. The juice is then fermented by the microbial mixture at a temperature of 22-33° C. for 6-15 days to form the fermented juice of group (i.e., a third control group) A4. Thus, it is possible of observing whether there is different effect between the group A1 and the group A4 using four different strains.

The groups A1 to A4 are compared with an unfermented juice of group A5 in terms of anti-oxidation ability and concentration of antioxidant in which a first experiment directed to evaluation of anti-oxidation ability and a second experiment directed to concentration of antioxidant are detailed below.

With respect to the first experiment directed to evaluation of anti-oxidation ability, ABTS (2,2′-Azino-bis-[3-ethylbenthiazoline sulfonic acid]) is a chromogenic agent. ABTS becomes ABTS+, a stable green substance absorbing light at 734 nm, when interacts with an oxidant in the presence of hydrogen peroxide (H₂O₂). Moreover, the presence of antioxidants can inhibit the formation of ABTS+. Therefore, the lower the absorbance measured at 734 nm, the greater anti-oxidation ability of the antioxidant.

Accordingly, 500-fold diluted fermented juices of groups A1 to A4 as well as 500-fold diluted unfermented juice of group A5 are used for measuring the ABTS+ clearance rate (%).

Referring to FIG. 1a, it is found that the experimental group A1 has an ABTS+ clearance rate (%) greater than each of the first, second and third control groups A2, A3 and A4 and the unfermented juice of group A5.

The groups A1 to A4 and the unfermented juice of group A5 are further diluted 500-fold. Vitamin C level and carotenoid level of the groups A1 to A5 are measured and its results are shown in FIGS. 1b and 1c. It is found that vitamin C level and carotenoid level of the experimental group A1 are greater than each of the first, second and third control groups A2, A3 and A4 and the unfermented juice of group A5.

In view of above, it is found that the experimental group A1 having the microbial mixture of L. casei strain, B. longum strain and S. cerevisiae strain has greater vitamin C level and carotenoid level than that of each of the first control group A2 having the microbial mixture of Lactobacillus acidophilus strain, B. longum strain and S. cerevisiae strain, the second control group A3 having the microbial mixture of Bifidobacterium bifidum strain, Saccharomyces boulardii strain and Lactobacillus acidophilus strain, and the third control group A4 having the microbial mixture of Lactobacillus casei strain, Bifidobacterium longum strain, Saccharomyces cerevisiae strain and Lactobacillus bulgaricus strain, and the unfermented juice of group A5.

It is concluded that the experimental group A1 has increased anti-oxidation ability and concentration of antioxidant in comparison with that of each of the first control group A2, the second control group A3, the third control group A4, and the unfermented juice of group A5.

The groups A1 to A4 and the unfermented juice of group A5 are further diluted 100-fold respectively in order to measure superoxidase dismutase (SOD) activity thereof. Results are shown in FIG. 1d. It is found that SOD activity of the experimental group A1 is significantly greater than each of the first, first and third control groups A2, A3 and A4 and the unfermented juice of group A5. It is further proved that the experimental group A1 has increased anti-oxidation ability in comparison with that of each of the first control group A2, the second control group A3, the third control group A4, and the unfermented juice of group A5.

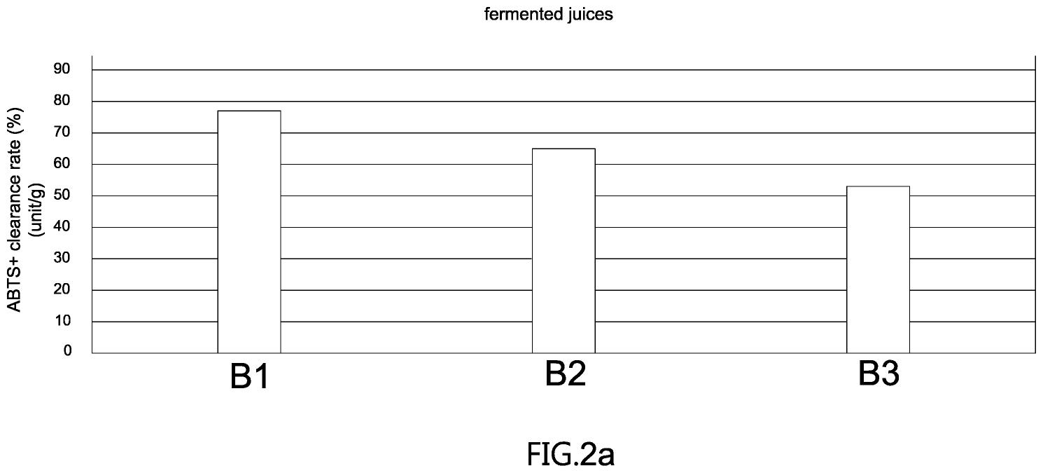

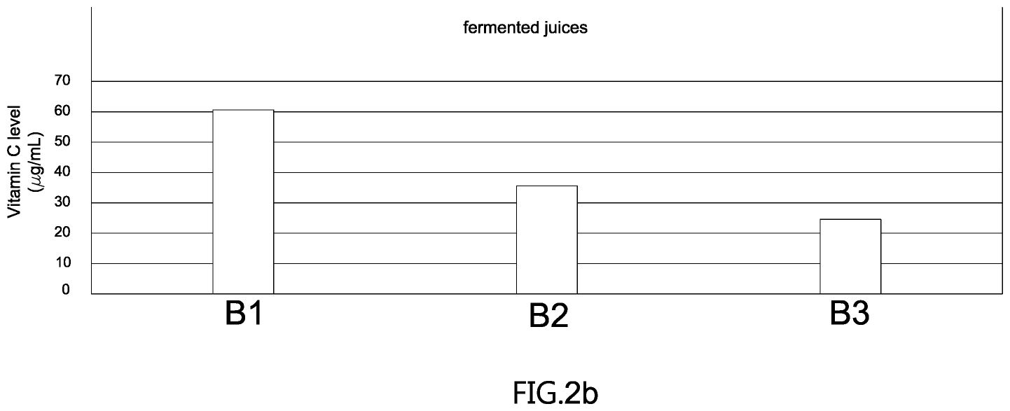

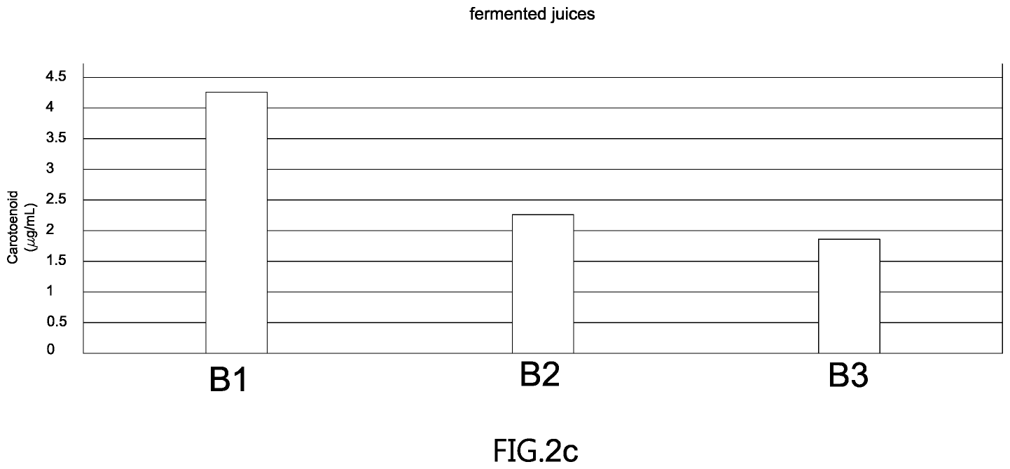

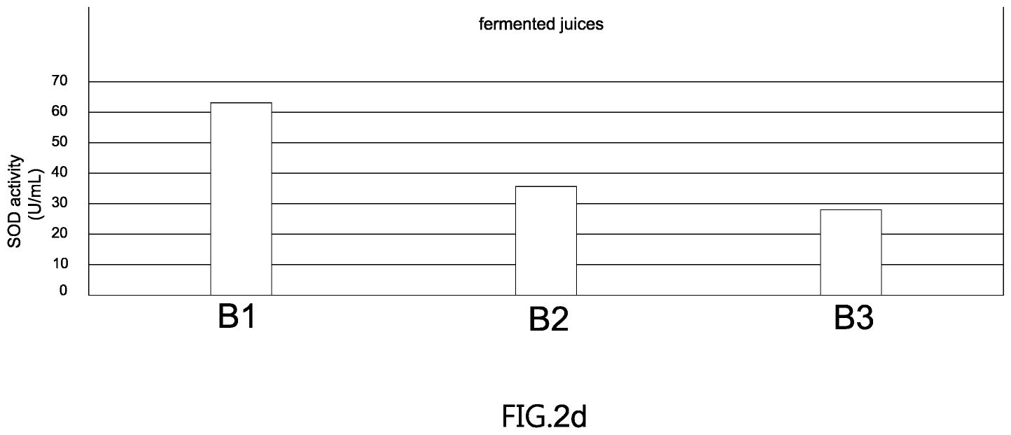

The second experiment is directed to evaluate inventive steps of the method of fermenting Rosa sterilis var. leioclada according to the invention in terms of concentration of strains. Results are shown and discussed below by referring to FIGS. 2a to 2d in which FIG. 2a depicts a bar chart illustrating ABTS+ clearance rate of the fermented juices of groups B1 to B2 as well as the unfermented juice of group B3 in experiment B; FIG. 2b depicts a bar chart illustrating vitamin C level of the fermented juices of groups B1 to B2 as well as the unfermented juice of group B3 in experiment B; FIG. 2c depicts a bar chart illustrating carotenoid level of the fermented juices of groups B1 to B2 as well as the unfermented juice of group B3 in experiment B; and FIG. 2d depicts a bar chart illustrating SOD activity of the fermented juices of groups B1 to B2 as well as the unfermented juice of group B3 in experiment B.

In the experiment B, the L. casei strain, the B. longum strain and the S. cerevisiae strain are mixed in the cell number ratio of 0.8:0.8:1.6 to obtain the microbial mixture. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at a temperature no greater than 28° C. for 14 days to form the fermented juice of group B1. In addition, the juice is fermented by the microbial mixture with the total concentration of the L. casei strain, the B. longum strain and the S. cerevisiae strain in the mixture solution being 1×10⁷ CFU/mL to form the fermented juice of group B2. The unfermented juice is used as group B3. Anti-oxidation ability (ABTS+ clearance rate and SOD activity) as well as antioxidants (vitamin C and carotenoid) level of the fermented juices of groups B1 and B2 and the unfermented juice of group B3 are measured.

Referring to FIGS. 2a and 2b, the fermented juice of group B1 has significantly improved ABTS+ clearance rate and significantly increased SOD activity. Referring to FIGS. 2c and 2d, the fermented juice of group B1 also has elevated antioxidant level such as vitamin C level and carotenoid level. That is, compared to fermenting the fruit sample by the microbial mixture with the total concentration of the L. casei strain, the B. longum strain and the S. cerevisiae strain in the mixture solution being 1×10⁷ CFU/mL, fermenting the fruit sample by the microbial mixture with the total concentration of the L. casei strain, the B. longum strain and the S. cerevisiae strain in the mixture solution being 3×10⁷ CFU/mL can effectively obtain the fermented juice with improved anti-oxidation ability.

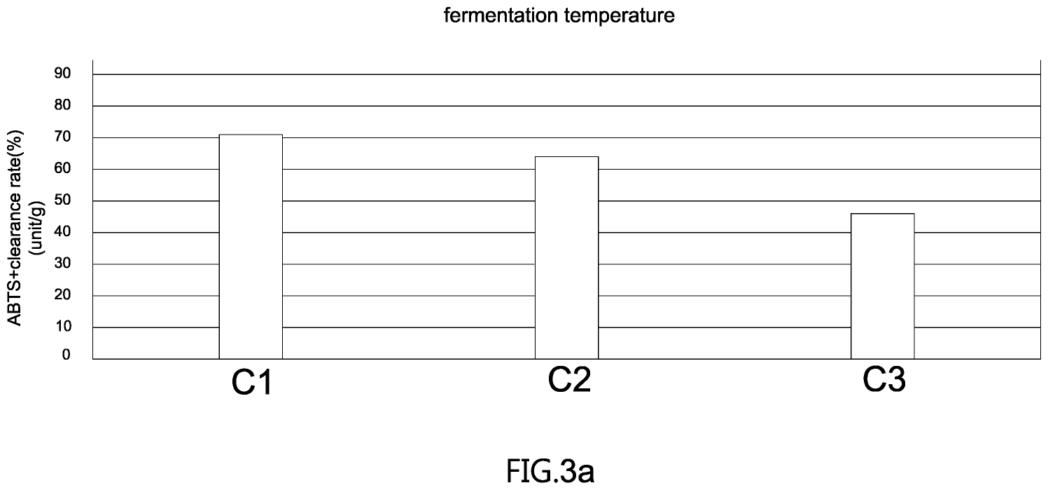

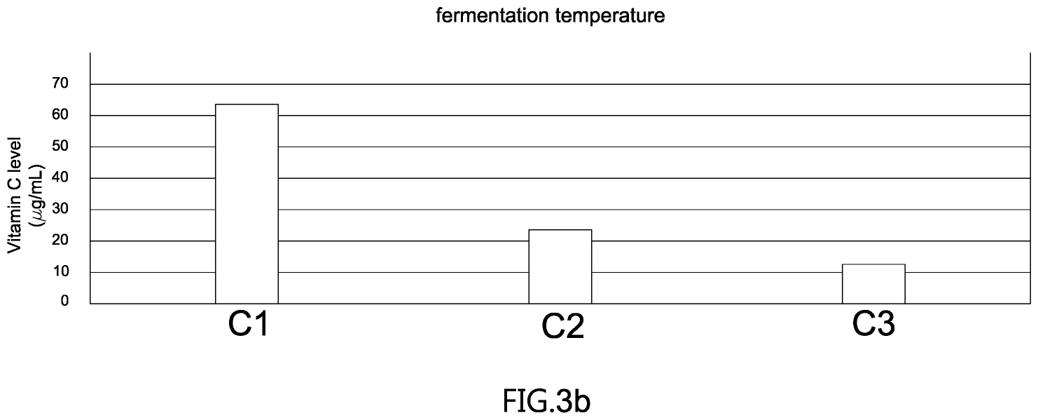

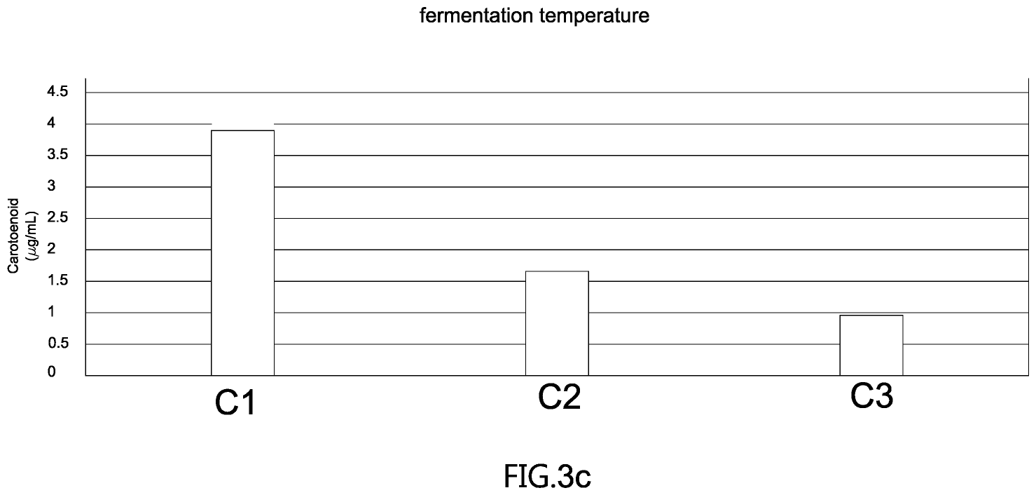

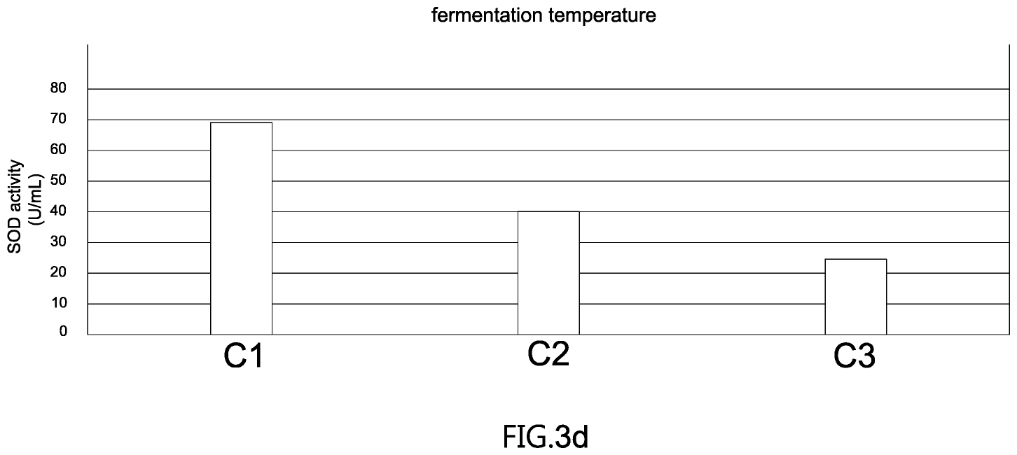

A third experiment is directed to evaluate inventive steps of the method of fermenting Rosa sterilis var. leioclada according to the invention in terms of fermentation temperature. Results are shown and discussed below by referring to FIGS. 3a to 3d in which FIG. 3a depicts a bar chart illustrating ABTS+ clearance rate of the fermented juices of groups C1 to C2 as well as the unfermented juice of group C3 in experiment C; FIG. 3b depicts a bar chart illustrating vitamin C level of the fermented juices of groups C1 to C2 as well as the unfermented juice of group C3 in experiment C; FIG. 3c depicts a bar chart illustrating carotenoid level of the fermented juices of groups C1 to C2 as well as the unfermented juice of group C3 in experiment C; and FIG. 3d depicts a bar chart illustrating SOD activity of the fermented juices of groups C1 to C2 as well as the unfermented juice of group C3 in experiment C.

In the experiment C, the L. casei strain, the B. longum strain and the S. cerevisiae strain are mixed in the cell number ratio of 0.8:0.8:1.6 to obtain the microbial mixture with. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at a temperature no greater than 28° C. for 14 days to form the fermented juice of group C1. In addition, the juice is fermented by the microbial mixture at 20° C. for 14 days to form the fermented juice of group C2. The unfermented juice is used as group C3. Anti-oxidation ability (ABTS+ clearance rate and SOD activity) as well as antioxidants (vitamin C and carotenoid) level of the fermented juices of groups C1 and C2 and the unfermented juice of group C3 are measured.

Referring to FIGS. 3a and 3b, the fermented juice of group C1 has significantly improved ABTS+ clearance rate and significantly increased SOD activity. Referring to FIGS. 3c and 3d, the fermented juice of group C1 also has elevated antioxidant level such as vitamin C level and carotenoid level. That is, compared to fermenting the fruit sample at 20° C., fermenting the fruit sample at a temperature no greater than 28° C. can effectively obtain the fermented juice with improved anti-oxidation ability.

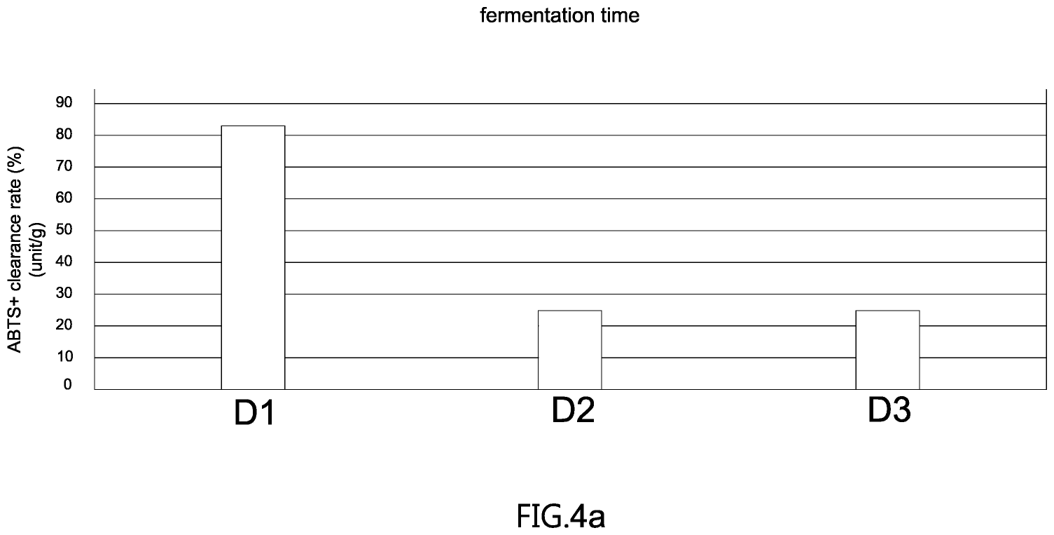

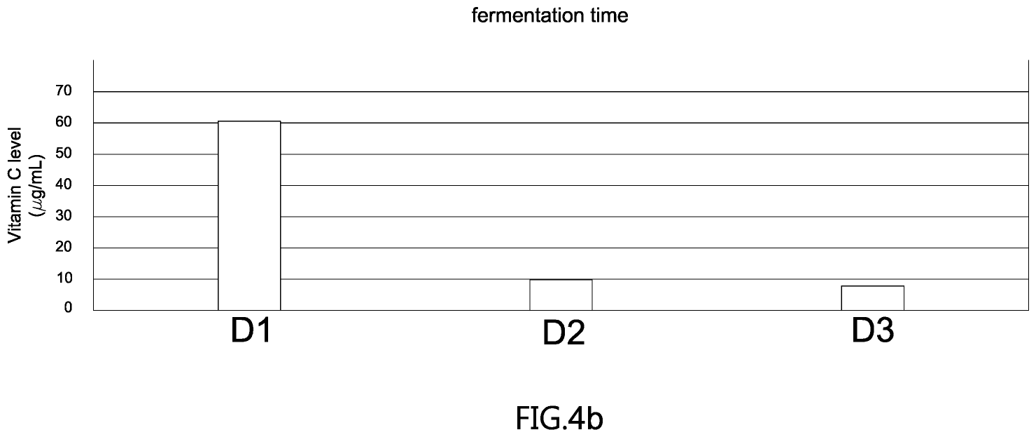

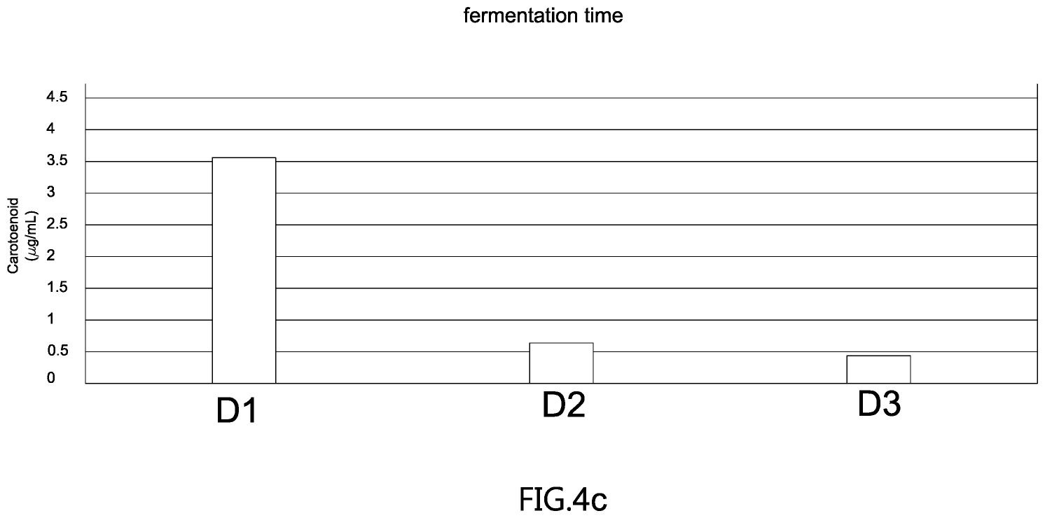

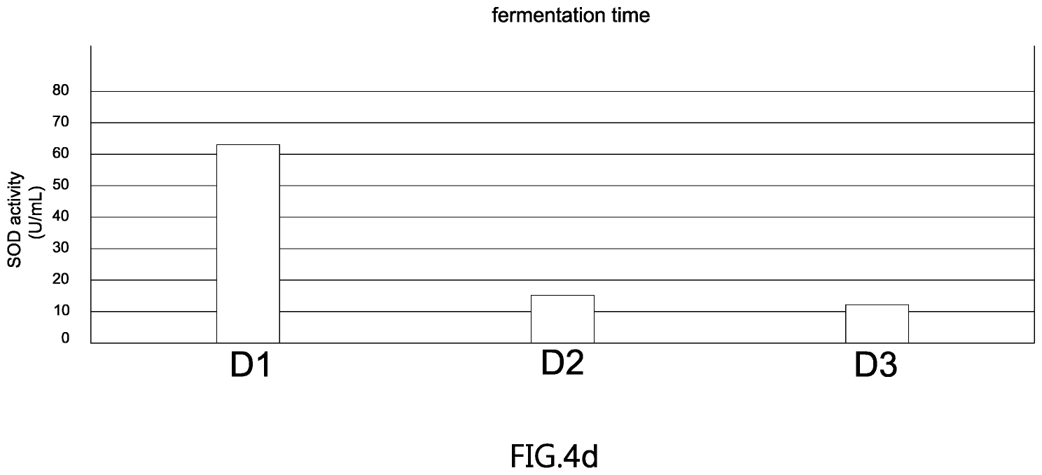

A fourth experiment is directed to evaluate inventive steps of the method of fermenting Rosa sterilis var. leioclada according to the invention in terms of fermentation time. Results are shown and discussed below by referring to FIGS. 4a to 4d in which FIG. 4a depicts a bar chart illustrating ABTS+ clearance rate of the fermented juices of groups D1 to D2 as well as the unfermented juice of group D3 in experiment D; FIG. 4b depicts a bar chart illustrating vitamin C level of the fermented juices of groups D1 to D2 as well as the unfermented juice of group D3 in experiment D; FIG. 4c depicts a bar chart illustrating carotenoid level of the fermented juices of groups D1 to D2 as well as the unfermented juice of group D3 in experiment D; and FIG. 4d depicts a bar chart illustrating SOD activity of the fermented juices of groups D1 to D2 as well as the unfermented juice of group D3 in experiment D.

In the experiment D, the L. casei strain, the B. longum strain and the S. cerevisiae strain are mixed in the cell number ratio of 0.8:0.8:1.6 to obtain the microbial mixture. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at a temperature no greater than 28° C. for 14 days to form the fermented juice of group D1. In addition, the juice is fermented by the microbial mixture at a temperature no greater than 28° C. for 5 days to form the fermented juice of group D2. The unfermented juice is used as group D3. Anti-oxidation ability (ABTS+ clearance rate and SOD activity) as well as antioxidants (vitamin C and carotenoid) level of the fermented juices of groups D1 and D2 and the unfermented juice of group D3 are measured.

Referring to FIGS. 4a and 4b, the fermented juice of group D1 has significantly improved ABTS+ clearance rate and significantly increased SOD activity. Referring to FIGS. 4c and 4d, the fermented juice of group D1 also has elevated antioxidant level such as vitamin C level and carotenoid level. Moreover, the fermented juice of group D2 shows similar anti-oxidation ability and antioxidants level to the unfermented juice of group D3. Thus, fermenting the fruit sample for 6-15 days can effectively obtain the fermented juice with improved anti-oxidation ability.

In conclusion, by fermenting the fruit sample by the microbial mixture including the L. casei strain, the B. longum strain and the S. cerevisiae strain, the obtained fermented juice has improved anti-oxidation ability. Therefore, the fermented juice can be used as a healthy product as well as an additive with anti-oxidation ability.

While the invention has been described in terms of preferred embodiments, those skilled in the art will recognize that the invention can be practiced with modifications within the spirit and scope of the appended claims.

What is claimed is:

1. A method of fermenting Rosa sterilis var. leioclada, comprising the steps of:
mixing a Lactobacillus casei strain, a Bifidobacterium longum strain, and a Saccharomyces cerevisiae strain to form a microbial mixture wherein a cell number ratio of the Lactobacillus casei strain, the Bifidobacterium longum strain and the Saccharomyces cerevisiae strain is 0.3-1:0.2-0.9:1-1.8;
preparing a fruit sample of Rosa sterilis var. leioclada; and
fermenting the fruit sample of Rosa sterilis var. leioclada with the microbial mixture at 22-33° C. for 6-15 days to obtain a fermented juice wherein a total concentration of the Lactobacillus casei strain, the Bifidobacterium longum strain, and the Saccharomyces cerevisiae strain in a mixture solution containing the microbial mixture and the fruit sample of Rosa sterilis var. leioclada is between 1.5×10⁷ and 3.5×10⁷ CFU/mL; and a volumetric percentage of the microbial mixture is 10-25% of a volume of the mixture solution.

2. The method of claim 1, wherein a Lactobacillus casei strain BCRC 10697, a Bifidobacterium longum strain BCRC 14604, and a Saccharomyces cerevisiae strain BCRC 20579 are mixed to form the microbial mixture.

3. The method of claim 1, wherein the fruit sample of Rosa sterilis var. leioclada is fermented by the microbial mixture at a temperature no greater than 28° C.

4. The method of claim 1, wherein the fruit sample of Rosa sterilis var. leioclada is fermented by the microbial mixture for 14 days.
